(19) United States
(12) Patent Application Publication
(10) Pub. No.: US 2010/0152584 A1
(43) Pub. Date: Jun. 17, 2010

(54) METHOD FOR MEASURING VARIOUS PARAMETERS OF BONES AND JOINTS

(76) Inventors: Arie Ariav, Doar-Na Hof Ashkelon (IL); Vladimir Ravitch, Ashkelon (IL)

Correspondence Address: MARTIN D. MOYNIHAN d/b/a PRTSI, INC., P.O. BOX 16446, ARLINGTON, VA, 22215, US

(21) Appl. No.: 12/374,676

(22) PCT Filed: Jul. 25, 2007
(86) PCT No.: PCT/IL07/00935

§ 371 Date: Feb. 2, 2010

Related U.S. Application Data
(60) Provisional application No. 60/832,949, filed on Jul. 25, 2006.

(51) Int. Cl. A61B 8/00

(52) U.S. Cl. 600/442

(57) ABSTRACT

A method of measuring a predetermined parameter of interest in a bone or joint of a subject's body, comprising embedding in the bone or joint an acoustical transmitter and an acoustical receiver spaced from the transmitter to define between them an acoustical transmission channel which includes a portion of the bone or joint exhibiting the predetermined parameter of interest, measuring the transit time of an acoustical wave transmitted through the acoustical transmission channel from the transmitter to the receiver, and utilizing the measured transit time to provide a measurement of the predetermined parameter of interest.

### FIELD AND BACKGROUND OF THE INVENTION

The present invention relates to a method for measuring various parameters of bones and joints. The invention is particularly useful in measuring certain parameters of interest in a knee joint, and is therefore described below with respect to such application, but it will appreciated the invention may be used with respect to other joints, e.g., the hip joint, or with respect to other bones in the human body.

It is frequently necessary or desirable to measure certain parameters of a bone or of a joint, such as the chemical composition of bone structure in the joint; changes of bone density or flexibility; loading at the bone or joint; mineralization or ossification of a portion of the bone or joint; deformation of a joint implant; friction, smoothness, heat-generation, or inflammation at a bone joint; gap at the bone joint, etc. Various ultrasonic techniques have been proposed for this purpose, as described for example in U.S. Pat. No. 6,468,215. However, in such known techniques, the measurements are effected by sensors located externally of the body, and therefore frequently lack the precision or sensitivity that may be required for particular types of measurements.

### OBJECTS AND BRIEF SUMMARY OF THE PRESENT INVENTION

An object of the present invention is to provide a method of measuring various parameters of bones or joints, which method is capable of a high degree of precision and sensitivity, and/or coverage of areas of interest.

According to a broad aspect of the present invention, there is provided a method of measuring a predetermined parameter of interest in a bone or joint of a subject's body, comprising: embedding in the bone or joint an acoustical transmitter and an acoustical receiver spaced from the transmitter to define between them an acoustical transmission channel which includes a portion of the bone or joint exhibiting the predetermined parameter of interest; measuring the transit time of an acoustical wave transmitted through the acoustical transmission channel from the transmitter to the receiver; and utilizing the measured transit time to provide a measurement of the predetermined parameter of interest.

According to further features in the described preferred embodiments, the method further comprises also embedding in, or in the vicinity of, the bone or joint an RF transmitter for transmitting the measurement of the transit time or predetermined parameter to a receiver located externally of the subject's body.

According to a still further feature in the described preferred embodiments, the transit time is measured by changing the frequency of the acoustical wave transmitted by the transmitter to the receiver via the acoustical transmission channel, while maintaining the number of waves in a loop including the acoustical transmission channel as a whole integer irrespective of changes in the predetermined parameter of interest; and utilizing the changes in frequency to produce a measurement of the transit time.

According to the described preferred embodiments, the acoustical transmitter, acoustical receiver, and RF transmitter are all incorporated in an implantable sensor which also includes an integrated circuit and a battery power supply for powering the sensor.

According to another aspect of the present invention, there is an implantable sensor unit particularly useful for measuring a predetermined parameter of interest in a bone or joint of a subject's body, comprising an acoustical transmitter, an acoustical receiver spaced from the transmitter to define between them an acoustical transmission channel which includes a portion of the bone or joint exhibiting the predetermined parameter of interest; an integrated circuit; and a battery power supply for powering the sensor.

As will be described more fully below, such a method is capable of measuring the transit time of an acoustical wave with an extremely high degree of precision. Accordingly, the method is capable of measuring virtually any parameter having a known or predetermined influence on either the transit distance and/or the transit velocity of an acoustical wave.

The description below refers to many parameters of bones and joints which can be measured in this manner, including: the temperature of the portion of the bone or joint in the acoustical transmission channel, the presence of cracks or wounds in the portion of the bone or joint in the acoustical transmission channel, the smoothness of the portion of the bone or joint in the acoustical transmission channel, the presence of physical stresses in the portion of the bone or joint in the acoustical transmission channel, changes in bone density and/or flexibility in the portion of the bone or joint in the acoustical transmission channel, movements of the portion of the bone join in the acoustical transmission channel, and the velocity of blood through a blood vessel in the portion of the bone or joint in the acoustical transmission channel.

The invention of capable of being implemented in a natural bone or joint of the subject, wherein the transducers and electronics involved can be embedded by open surgical procedure, by arthroscopic or other minimally invasive procedure, or by injection of the transducers and electronic components into the vicinity of the bone or joint. The invention, however, is particularly useful when implemented in artificial bone or joints, since the components of the sensors, including an RF transmitter, can be incorporated in the artificial bone or joint before implanted in the subject.

As indicated earlier, the invention is particularly useful for measuring various parameters of knee joints, and is therefore described below with respect to that application.

Further features and advantages of the invention will be apparent from the description below.

### BRIEF DESCRIPTION OF THE DRAWINGS

The invention is herein described, by way of example only, with the reference to the accompanying drawings, wherein:

FIG. 1 schematically illustrates a normal knee joint in which has been embedded the acoustical transmitter and acoustic receiver, together with the electronics involved, to measure one or more predetermined parameters of interest in the bone or joint of a subject's body;

FIG. 2 is a schematic diagram of the electronic components embedded in the knee joint, together with RF antenna for transmitting the sensed measurements to an external device;

FIGS. 3, 4, 5 and 6 illustrate various artificial bone or joints equipped with various arrangements of acoustical transducers and receivers for measuring different predetermined parameters in the knee joint of a subject's body;

FIG. 7 illustrates an implantable sensor constructed in accordance with the present invention; and

FIG. 8 schematically illustrates the internal construction of the implantable sensor of FIG. 7.

It is to be understood that the foregoing drawings, and the description below, are provided primarily for purposes of facilitating understanding the conceptual aspects of the invention and possible embodiments thereof, including what is presently consider

### DESCRIPTION OF PREFERRED EMBODIMENTS

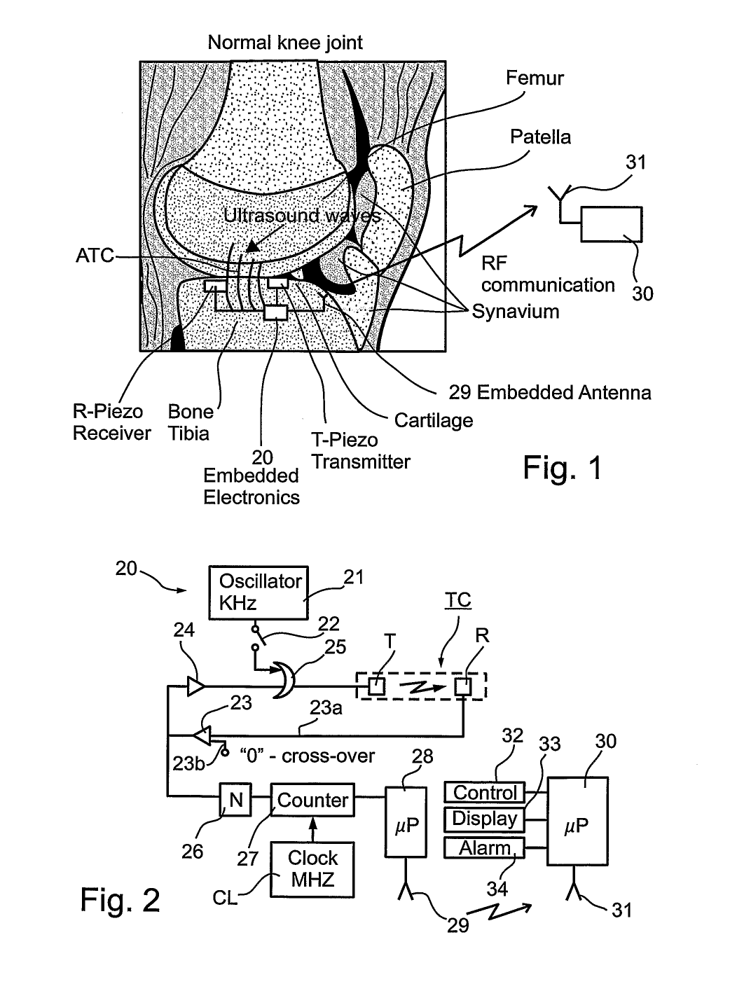

FIG. 1 schematically illustrates a normal knee joint, and one manner of measuring in accordance with the present invention various parameters of interest with respect to the knee joint. Shown in FIG. 1 are three of the bones at the knee joint, including: the femur, which is the large bone in the thigh, the tibia, which is the leg bone attached to the femur by ligaments and a capsule, and the patella, the knee cap, which rides on the knee joint as the knee bends. The fourth bone in the knee joint, namely the fibula which runs parallel to the tibia, is not shown in FIG. 1.

In accordance with the present invention, embedded in the knee joint, particularly the tibia, are an acoustical transmitter T and an acoustical receiver R spaced from the transmitter to define between them an acoustical transmission channel ATC which includes a portion of the bone or joint manifesting the one or more predetermined parameters of interest. In this case, and as shown in FIG. 1; the acoustical transmission channel ATC between the transmitter T and the receiver R is the surface of the tibia facing the femur.

Also embedded in the knee joint is the electronics unit, generally designated 20, for measuring the transit time of an acoustical wave transmitted through the acoustical transmission channel ATC from the transmitter T to the receiver R, and for utilizing the measured transit time to provide a measurement of the one or more predetermined parameters of interest. The electronic unit 20 in FIG. 1 is more particularly illustrated in FIG. 2, and the manner in which it utilizes the transmitter T and receiver R for measuring the predetermined parameters of interest is described below. The embedded electronics further include an antenna 29 which transmits the measurements, or the data pertaining to the measurements, to an external unit 30 having an antenna 31 receiving the transmissions from the embedded antenna 29.

The system for measuring the transit time of an acoustical wave through the acoustical transmission channel ATC, namely from the transmitter T to receiver R, is preferably the one described in our prior U.S. Pat. No. 6,621,278. Such a circuit is capable of producing a precise measurement of the transit time of an acoustical wave through the acoustical transmission channel ATC, and therefore a very precise measurement of one or more predetermined parameters of interest influencing such transit times.

Briefly, the system illustrated in FIG. 2 operates by: (a) transmitting from transmitter T a cyclically-repeating energy wave through the acoustical transmission channel ATC defined with receiver R; (b) changing the frequency of the transmission while maintaining the number of waves in the loop including the acoustical transmission channel as a whole integer; and (c) utilizing the changes in frequency of the transmission to provide an indication of the deformation of the force applied.

In the described preferred embodiment, operation (b) includes: detecting a predetermined fiducial point in each cyclically-repeating energy wave received by receiver R; and continuously changing the frequency of the transmission in accordance with the detected fiducial point of each received energy wave such that the number of energy waves in the loop of the transmission channel is a whole integer.

More particularly, the system illustrated in FIG. 2 operates as follows: Initially, oscillator 21 is energized while switch 22 is closed so as to cause transmitter 4 to transmit a succession of sonic pulses until such pulses are received by receiver R. Once the pulses are received by receiver R, switch 22 is opened so that the pulses received by receiver R are thereafter used for controlling the transmitter T.

As shown in FIG. 2, the sonic signals received by receiver R are fed to a comparator 23 via its input 23a. Comparator 23 includes a second input 23b connected to a predetermined bias so as to detect a predetermined fiducial or reference point in the received signal. In the example illustrated in FIG. 2, this predetermined fiducial point is the “zero” cross-over point of the received signal; therefore, input 23b of comparator 23 is at a zero bias.

The output of comparator 23 is fed to an amplifier 24, e.g., a monostable oscillator, which is triggered to produce an output signal at each fiducial point (zero cross-over point) in the signals received by receiver R. The outputs from amplifier 24 are fed via an OR-gate 25 to trigger the transmitter T for the next sonic pulse. Since switch 22 is open, transmitter T will thus be triggered by each signal received by the receiver R to transmit the next sonic pulse in the succession of pulses.

It will thus be seen that the frequency of the output pulses or signals from transmitter T will change with a change in the spacing (transit distance) between the transmitter T and receiver R, and/or a change in the transit velocity. It will also be seen that the number of wavelengths or pulses in the loop including transmitter T and receiver R will be a whole integer. This change in frequency by the transmitter T, while maintaining the number of waves between the transmitter and receiver R as a whole integer, enables a precise determination to be made of the distance between the transmitter and receiver and/or the transit velocity, and thereby of the deformation of membrane 11.

A summing circuit, including counter 26, counter 27, clock CL and microprocessor 28, enables the detected frequency difference, and thereby the measurement precision, to be increased by a factor “N”. Thus, the precision of the measurement can be preset, almost without limitation, by the selection of the appropriate frequency, clock rate for clock CL, and summation factor “N” for counter 27.

As further shown in FIG. 2, the output from microprocessor 28 is fed to the embedded antenna 29 for transmission to an external unit, schematically indicated at 30, via its receiving antenna 31. External unit 30 may also include a microprocessor for processing the information communicated to it from the embedded sensor, and for providing a measurement of the one or more predetermined parameters of interest. As further shown in FIG. 2, the output from external unit 30 may be used for control, display, and/or alarm purposes, as schematically shown by blocks 32, 33 and 34.

Further details of the construction and operation of such a measuring circuit are described in the above-cited U.S. Pat. No. 6,621,278, the contents of which are incorporated by reference.

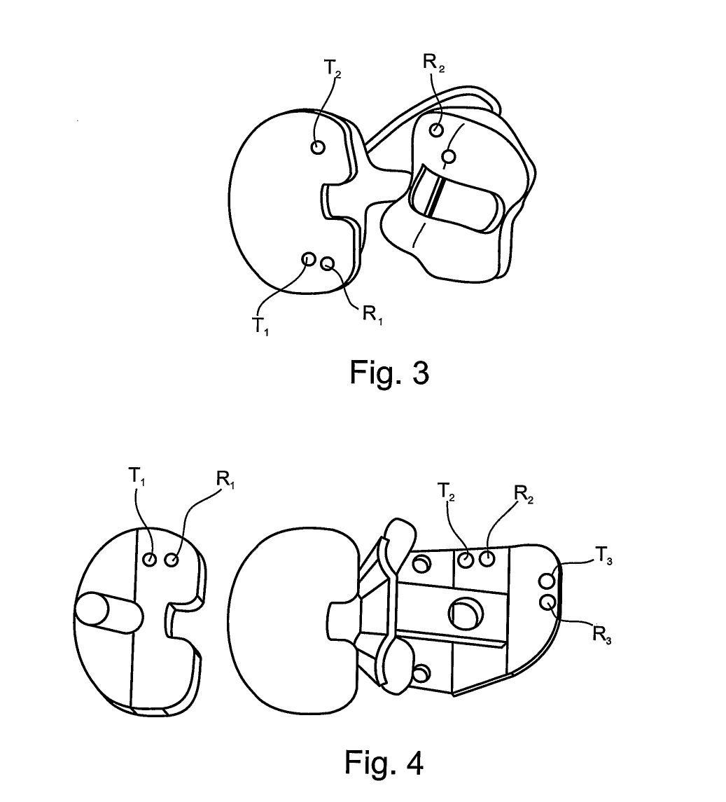

FIG. 3 illustrates a knee implant and possible arrangements of sensors for measuring various predetermined parameters of interest in the knee implant. Thus, the knee implant illustrated in FIG. 3 includes a first acoustical transmission channel defined by a first transmitter T₁ and receiver R₁ in one part of the implant for measuring the temperature, presence of cracks or wounds, smoothness, the presence of physical stresses, changes in bone density and/or flexibility, or the like in the respective part of the knee implant. The illustrated knee implant includes a second acoustical transmission channel defined by a transmitter T₂ on one part of the knee implant and a receiver R₂ on a different part of the knee implant, e.g., useful for measuring friction, or movements, or the like, with respect to the two parts of the joint defined by the knee implant.

FIG. 4 illustrates a knee implant which includes a plurality of separate acoustical transmission channels, each defined by a transmitter and a receiver, e.g., for measuring density of the bone area in the respective part of the acoustical transmission channel. Thus, FIG. 4 illustrates a first transmission channel defined by transmitter T₁ and receiver R₁ on one part of the knee implant, and two further transmission channels, defined by transmitter T₂ and receiver R₂, and transmitter T₃ and receiver R₃, at different portions on another part of the knee joint. It will be appreciated that the arrangement illustrated in FIG. 4 is capable of measuring, not only changes in density in the respective portion of the knee implant defined by the respective acoustical transmission channel, but also the presence of cracks or wounds, smoothness, the physical stresses, and the flexibility in the respective of the knee implant.

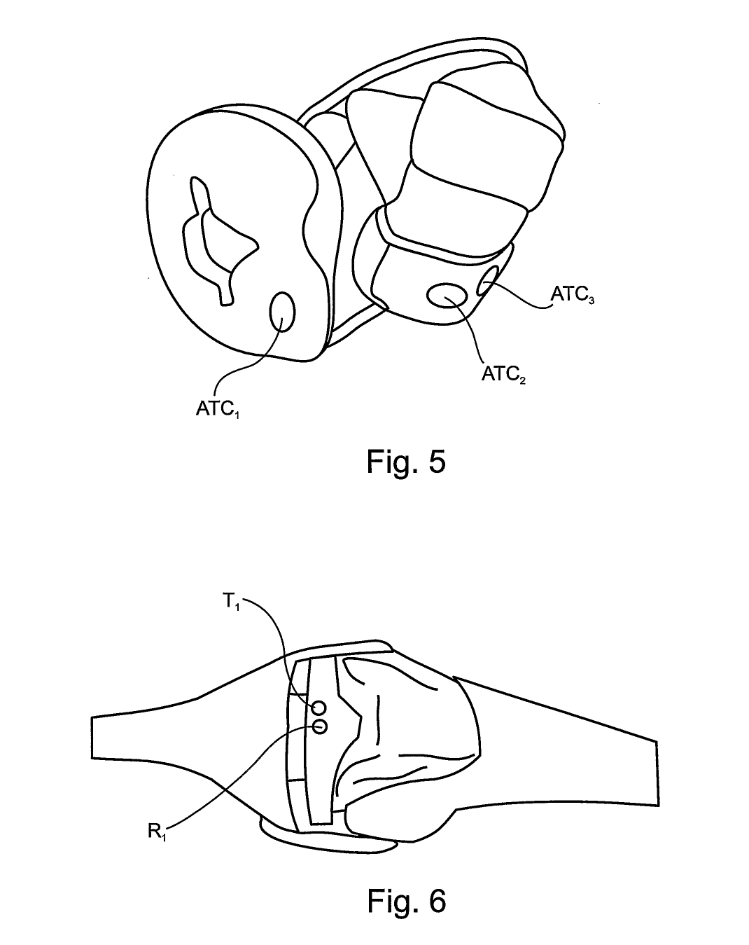

FIG. 5 illustrates a knee implant also including a plurality of acoustical transmission channels for measuring a predetermined parameter of interest in the respective portion of the knee implant. It will be appreciated that, in the implant illustrated in FIG. 5, each acoustical transmission channel ATC₁, ATC₂ and ATC₃ includes a transmitter and a receiver as described above for measuring the particular parameter of interest which produces the change in transit distance and/or transit velocity precisely measured as described above.

FIG. 6 illustrates a further knee implant including a single acoustical transmission channel as defined by a transmitter T₁ and receiver R₁ for measuring the parameter of interest in the respective part of the implant occupied by the acoustical transmission channel. The arrangement illustrated in FIG. 6 may also be used for measuring various parameters of tissues surrounding the joint.

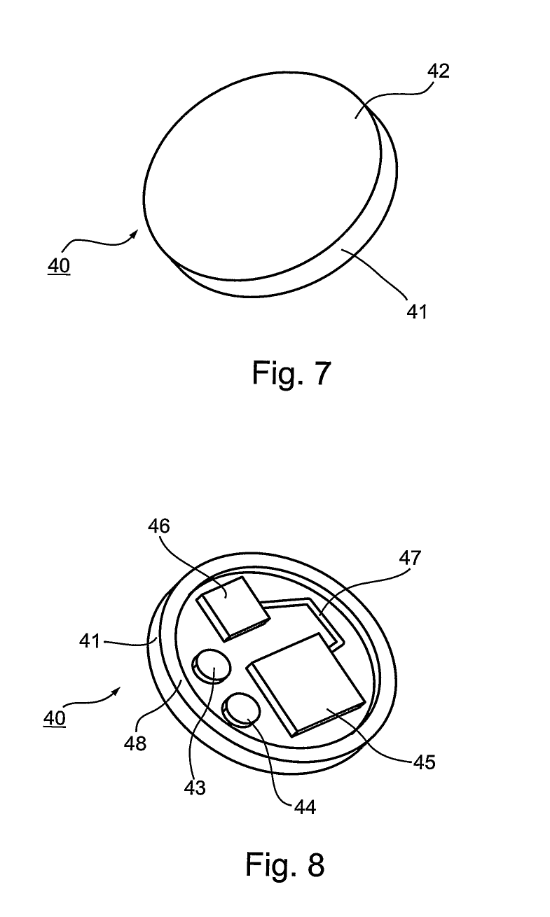

FIGS. 7 and 8 illustrate one form of implantable sensor that may be used in accordance with the present invention. The implantable sensor, generally designated 40 in FIGS. 7 and 8, includes a housing 41 of circular of pill-shaped configuration, closed by cover 42. The interior of housing 41 includes an acoustical transmitter 43 and an acoustical receiver 44 spaced from the transmitter to define between them an acoustical transmission channel which includes a portion of the bone, joint, or other tissue exhibiting the predetermined parameter of interest.

Implantable sensor 40 further includes an application-specified integration circuit (ASIC) constructed as schematically shown in FIG. 2 for producing the measurements described above, an RF transmitter 46 and an RF antenna 47 for transmitting the measurements to a receiver located externally of the subject's body, and a battery 48 for powering the sensor.

It will thus be seen that the invention can be used for measuring, with extremely high precision, virtually any parameter influencing the transit distance and/or transit velocity of an acoustical wave between the implanted transmitter and receiver. For example, besides the parameters referred to above, the acoustical transmission channel also includes a blood vessel, the velocity of the blood flowing through the blood vessel may also be measured in this manner.

While the invention has been described with respect to several preferred embodiments, it will be appreciated that these are set forth merely for purposes of example, and that many other variations, modifications and applications of the invention may be made.

1. A method of measuring a predetermined parameter of interest in a bone or joint of a subject's body, comprising:
embedding in the bone or joint an acoustical transmitter and an acoustical receiver spaced from said transmitter to define between them an acoustical transmission channel which includes a portion of the bone or joint exhibiting said predetermined parameter of interest;
measuring the transit time of an acoustical wave transmitted through said acoustical transmission channel from said transmitter to said receiver;
and utilizing said measured transit time to provide a measurement of said predetermined parameter of interest.

2. The method according to claim 1, further comprising also embedding in, or in the vicinity of, said bone or joint an RF transmitter for transmitting the measurement of said transit time or predetermined parameter to a receiver located externally of said subject's body.

3. The method according to claim 1, wherein said predetermined parameter of interest is temperature of the portion of the bone or joint in said acoustical transmission channel.

4. The method according to claim 1, wherein said predetermined parameter of interest is the presence of cracks or wounds in the portion of the bone or joint in said acoustical transmission channel.

5. The method according to claim 1, wherein said predetermined parameter of interest is the smoothness of the portion of the bone or joint in said acoustical transmission channel.

6. The method according to claim 1, wherein said predetermined parameter of interest is the presence of physical stresses in the portion of the bone or joint in said acoustical transmission channel.

7. The method according to claim 1, wherein said predetermined parameter of interest is a change in bone density and/or flexibility in the portion of the bone or joint in said acoustical transmission channel.

8. The method according to claim 1, wherein said predetermined parameter of interest is a movement of the portion of the bone or joint in said acoustical transmission channel.

9. The method according to claim 1, wherein said portion of the bone or joint includes a blood vessel, and the predetermined parameter of interest is the velocity of the blood through said blood vessel.

10. The method according to claim 1, wherein said transit time is measured by changing the frequency of the acoustical wave transmitted by said transmitter to said receiver via said acoustical transmission channel, while maintaining the number of waves in a loop including said acoustical transmission channel as a whole integer irrespective of changes in said predetermined parameter of interest; and utilizing the changes in frequency to produce a measurement of said transit time.

11. The method according to claim 1, wherein said bone or joint is a natural bone or joint of the subject.

12. The method according to claim 1, wherein said bone or joint is an artificial bone or joint implanted in the subject.

13. The method according to claim 1, wherein said bone or joint is a knee joint of the subject.

14. The method according to claim 2, wherein said acoustical transmitter, acoustical receiver, and RF transmitter are all incorporated in an implantable sensor which also includes an integrated circuit and a battery power supply for powering said sensor.

15. An implantable sensor unit particularly useful for measuring a predetermined parameter of interest in a bone or joint of a subject's body, comprising
an acoustical transmitter, an acoustical receiver spaced from said transmitter to define between them an acoustical transmission channel which includes a portion of the bone or joint exhibiting the predetermined parameter of interest; an integrated circuit; and a battery power supply for powering said sensor.

16. The sensor unit according to claim 15, wherein said unit further comprises an RF transmitter for transmitting measurements of the transit time of acoustical waves through said acoustical transmission channel to a receiver located externally of the subject's body.

17. An implantable system for measuring a predetermined parameter of interest in a bone or joint of a subject's body, comprising:
an acoustical sensor including an acoustical transmitter and an acoustical receiver designed and dimensioned to be embedded in the bone or joint in spaced relationship from each other to define between them an acoustical transmission channel which includes a portion of the bone or joint exhibiting said predetermined parameter of interest;
and an electronic control system for controlling the operation of said acoustical transmitter and acoustical receiver to measure the transit time of acoustical waves through said acoustical transmission channel, and for utilizing the measured transit time to provide a measurement of the predetermined parameter of interest.

18. The system according to claim 17, wherein said system further comprises an outer transmitter designed and dimensioned also to be embedded also in said bone or joint for transmitting the measurements of said electronic control system;
and an RF receiver designed to be located externally of the subject's body for receiving the transmissions of said RF transmitter.

19. The system according to claim 17, wherein said transit time is measured by changing the frequency of the acoustical wave transmitted by said transmitter to said receiver via said acoustical transmission channel, while maintaining the number of waves in a loop including said acoustical transmission channel as a whole integer irrespective of changes in said predetermined parameter of interest; and utilizing the changes in frequency to produce a measurement of said transit time.

20. The system according to claim 17, wherein said acoustical transmitter, acoustical receiver, and RF transmitter are all incorporated in an implantable sensor which also includes an integrated circuit and a battery power supply for powering said sensor.
